(19) United States
(12) Patent Application Publication
(10) Pub. No.: US 2013/0267008 A1
(43) Pub. Date: Oct. 10, 2013

(54) FREEZING MEDIUM COMPOSITION FOR CRYOPRESERVING AMNIOTIC FLUID-DERIVED STEM CELLS AND A METHOD FOR CRYOPRESERVING THE SAME

(71) Applicants: Yun-Hee Shon, Daegu (KR); James J. Yoo, Winston-Salem, NJ (US); Jang-Soo Suh, Daegu (KR)

(72) Inventors: Yun-Hee Shon, Daegu (KR); James J. Yoo, Winston-Salem, NJ (US); Jang-Soo Suh, Daegu (KR)

(73) Assignee: KYUNGPOOK NATIONAL UNIVERSITY HOSPITAL, Daegu (KR)

(21) Appl. No.: 13/578,972

(22) PCT Filed: Jan. 14, 2011
(86) PCT No.: PCT/KR11/00279

§ 371 Date: Aug. 14, 2012

(30) Foreign Application Priority Data
Dec. 31, 2010 (KR) 10-2010-0140278

(51) Int. Cl. C12N 5/0735

(52) U.S. Cl. CPC C12N 5/0606
USPC 435/192; 435/374

(57) ABSTRACT

Provided are to a freezing medium composition for cryopreserving amniotic fluid-derived stem cells, which has A lower concentration of Me₂SO and eliminates fetal bovine serum and at the same time does not induce cryoinjury to fluid-derived stem cells and makes it possible to cryopreserve fluid-derived stem cells for a prolonged time using trehalose, sucrose and catalase, and a method for cryopreserving the same. According to the present invention, AFSCs can be cryopreserved with 1/4 the standard Me2SO concentration with the addition of disaccharides, antioxidants and caspase inhibitors. As a result, the use of Me2SO at low concentrations in cell freezing solutions may support the development of clinical trials of AFSCs.

### TECHNICAL FIELD

This invention relates to a freezing medium composition for cryopreserving amniotic fluid-derived stem cells and a method for cryopreserving the same. More particularly, this invention relates to a freezing medium composition for cryopreserving amniotic fluid-derived stem cells, which has a reduced concentration of Me₂SO and is free of fetal bovine serum and at the same time does not induce cryoinjury to fluid-derived stem cells, thereby cryopreserving fluid-derived stem cells for a prolonged time using trehalose, sucrose and catalase, and a method for cryopreserving the same.

### BACKGROUND ART

Amniotic fluid (AF) has recently emerged as a potential source of well-characterized mesenchymal stem cells that can be obtained without raising the ethical concerns associated with human embryonic stem cell research. Amniotic fluid-derived stem cells (AFSCs) have been shown to differentiate into multiple cell lineages including adipose, bone, muscle and neural cells.

AF, which is usually discarded after a birth, could provide a more abundant source of stem cells than any other part of the human body. AFSCs have shown better growth rates and increased differentiation potential compared to adult bone marrow-derived stem cells. Furthermore, AFSCs display immunomodulatory properties, and thus, they could potentially be utilized in immunemediated disorders as well as in the treatment of graft-versus-host disease. Therefore, sourcing stem cells from AF would be relatively easy, and long-term banking of AFSCs would have a significant impact on future regenerative medicine technologies. However, one major obstacle to manufacturing clinical grade stem cells has been a lack of current good manufacturing practices in cell processing, cryopreservation, storage and distribution.

Developing effective techniques for the cryopreservation of AFSCs is an important step in the banking of stem cells. The freezing rate is a significant factor in determining the cell viability following cryopreservation and storage. Cooling the cells at a slow, controlled rate avoids intracellular ice buildup, which can cause the cell membrane to rupture. However, even slow freezing can result in dehydration of the cells by formation of extracellular ice, and for this reason, a cryoprotective agent is usually added to the freezing medium to prevent this.

The most widely used cryoprotectant (CPA) is dimethylsulfoxide (Me₂SO), which is a hygroscopic polar compound and can be toxic to cells. For example, the cryopreservation method used for AFSCs that is most commonly employed includes a freezing medium consisting of 10% Me₂SO as a CPA in the presence of either animal or human serum. However, there have been no studies that have investigated the clinical effects of the presence of Me₂SO in the cryopreservation media used for AFSCs despite the fact that transplantation of Me₂SO-cryopreserved hematopoietic stem cell products is frequently associated with serious side effects, such as vomiting, hypotension, acute abdominal pain, dyspnea, cardiac arrhythmia, and hemoglobinuria.

The addition and removal of these CPAs are complex processes associated with detrimental osmotic shock to the cells. Therefore, it would be valuable to develop CPA-free media or non-toxic CPAs for the cryopreservation and storage of stem cells. It remains to be seen how reduced concentrations of Me₂SO will affect the viability and performance of AFSCs frozen in either allogeneic/autologous serum or serum-free conditions.

Disaccharides, such as sucrose and trehalose, have been widely used as natural cryoprotectants, as well as excipients for freeze drying and as stabilizers during dehydration processes. In nature, many organisms have the ability to survive almost complete dehydration. This is a phenomenon known as anhydrobiosis, which is similar to the dehydration that occurs during cryopreservation. Anhydrobiosis is related in some instances to the accumulation of large amounts of disaccharides, such as trehalose, and this has sparked tremendous interest in the use of trehalose as a non-toxic CPA. For instance, L. S. Limaye, V. P. Kale, Cryopreservation of human hematopoietic cells with membrane stabilizers and bioantioxidants as additives in the conventional freezing medium, J. Hematother, Stem Cell Res. 10 (2001) 709-718 showed that trehalose was effective in preserving hematopoietic progenitor cells, and C. Scheinkonig, S. Kappicht, H. J. Kolb, M. Schleuning, Adoption of long-term cultures to evaluate the cryoprotective potential of trehalose for freezing hematopoietic stem cells, Bone Marrow Transplant. 34 (2004) 531-536 evaluated trehalose with insulin for the preservation of the colony forming capability of bone marrow and peripheral blood stem cells.

In addition to dehydration, formation of oxygen free radicals is another cause of loss of cell viability during or just after freezing. Limaye et al. showed that addition of bioantioxidants into the cryopreservation solution increases post-thaw recovery of cells. The use of membrane stabilizers and bioantioxidants has been shown to improve cryoprotection of human hematopoietic cells.

Another prior art shows that serum proteins used in the cryopreservation media are difficult to remove during washing, and residue left in the cell solution can trigger adverse reactions in patients who receive cell infusions or transplants. Therefore, the development of serum free media for the storage of stem cells meant for clinical use is a critical issue.

Among prior arts, Korean patent application No. 10-2006-7004375 ‘Biodegradable polymer-ligand conjugates and their use in isolation of cellular subpopulations and in cryopreservation, culture and transplantation of cells’ discloses a method of cryopreserving ankorage-dependent cell, containing steps of (a) forming mixture by anchoring cells to a composition containing at least a degradable particulate, (b) freezing the mixture, and (c) thawing and recovering cells from the cells-polymer particulate conjugates,

Korean patent application No. 10-2009-7007620 ‘Method for freeze preservation of tissue-derived cell’ discloses a method of cryopreserving tissue-derived cells, containing steps of fragmentating tissue including target cells, culturing the fragmentated tissue slices in a medium, recovering the cultured tissues slices and suspending them into a cryopreserving solution, and freezing the suspension of tissue slices at a temperature below −70 ° C., and

Korean Patent No. 1005342150000 ‘Method of isolating and culturing mesenchymal stemcell derived from cryopreserved umbilical cord blood’ discloses a method of isolating and culturing mesenchymal stemcell derived from cryopreserved umbilical cord blood, containing steps of thawing cryopreserved umbilical cord blood, diluting it with αMEM(alpha-minimum essential medium), centrifuging it to obtain monocytes; separating CD133 positive cells from the obtained monocytes; and suspension-culturing the separated cells into αMEM medium containing Stem Cell Factor, GM-CSF (granulocyte-macrophage colony-stimulating factor), G-CSF (granulocyte colony-stimulating factor), IL-3 (interleukin-3) and IL-6 (interleukin-6).

However, there is no patent or patent application disclosing a composition for cryopreserving amniotic fluid-derived stem cells and a method for cryopreserving the same.

### DISCLOSURE OF INVENTION

### Technical Problem

So far, one major obstacle to manufacturing clinical grade stem cells has been that there is no proper composition or method for cryopreserving, storing and distributing these cells. The latest cryopreservation composition and method used for stem cells contains toxic dimethyl sulfoxide (Me₂SO) as a cryoprotectant(CPA) in the presence of animal serum protein in the use of treatment of human beings. In order to prevent cryoprotectant-related complications, it is necessary to develope nontoxic CPA or reduce a concentration of CPA in a freezing medium.

### Solution to Problem

We, the present inventors, demonstrated that the major cause of loss of viability of human embryonic stem cells after slow freezing is apoptosis induced by cryoinjury. Preclinical data also suggests that activation of caspases during the freezing and thawing processes can induce apoptosis and hence contribute to cryoinjury in grafts for transplantation. Therefore, the use of caspase inhibitors in combination with other cryoprotective agents might be protective during the long-term storage of living cells, which is critical for the success of tissue engineering strategies.

Therefore, in the present invention, we found that a freezing medium containing disaccharides, bioantioxidants and caspase inhibitors would be useful in the preservation of AFSC. In order to test this hypothesis, we created various freezing media containing these components and tested them in a freezing protocol to see whether they provided enhanced protection for AFSCs and allowed us to reduce the final concentration of Me₂SO present in the final AFSC-containing infusion product.

Accordingly, the present invention provides a freezing medium composition for cryopreserving amniotic fluid-derived stem cells, characterized in that it contains trehalose, catalase and zVAD-fmk with a reduced concentration of Me₂SO.

Further, the present invention provides the freezing medium composition, characterized in that the concentration of Me₂SO is 2.5 to 5% (v/v) and a concentration of trehalose is 60 mmol/L and a concentration of catalase is 100 mg/mL and a concentration of zVAD-fmk is 30 μM.

In addition, the present invention provides a method of cryopreserving AFSC using the freezing medium composition.

### Advantageous Effects of Invention

According to the present invention, a statistically significant (p<0.05) increase in post-thawed cell viability in solutions containing trehalose, catalase and zVAD-fmk with 5% Me₂SO was observed. The solutions containing trehalose and catalase with 5% or 2.5% (v/v) Me₂SO produced results similar to those for the control (10% (v/v) Me₂SO and 30% FBS) in terms of culture growth, expression of cell surface antigens and mRNA expression of stem cell markers in AFSCs cryopreserved for a minimum of 3 weeks. Thus, AFSCs can be cryopreserved with ¼ the standard Me₂SO concentration with the addition of disaccharides, antioxidants and caspase inhibitors. The use of Me₂SO at low concentrations in cell freezing solutions has the industrial effect of supporting the development of clinical trials of AFSCs.

### BRIEF DESCRIPTION OF DRAWINGS

FIG. 1 is a graph showing MTT assay of post-thawed AFSCs cryopreserved with different composition of Me₂SO, FBS, trehalose, catalase and zVAD- fmk(Benzyloxycarbonyl-alyl-alanyl-aspartyl-(O-methyl)-fluoromethyl-ketone). Data shown are mean values with bars indicating the SD of the mean (n=3). *p<0.05 compared with the control.

FIG. 2 is culture growth curves of post-thawed AFSCs cryopreserved with different composition of Me₂SO, FBS, trehalose, catalase and zVAD-fmk.

FIG. 3 is graphs showing a flow cytometric analysis of post-thawed AFSCs cryopreserved in solution containing different composition of Me₂SO, FBS, trehalose and catalase.

FIG. 4 is images showing RT-PCR analysis of post-thawed AFSCs cryopreserved with different composition of Me₂SO, FBS, trehalose, catalase and zVAD-fmk, wherein Non-cryo meansnoncryopreserved AFSCs.

FIG. 5a is images showing western blot analysis of myogenic differentiation of post-thawed AFSCs cryopreserved in solution containing trehalose and catalase with 5% (solution 3) and 2.5% (v/v) Me₂SO (solution 6) after myogenic pathway induction with 5-azaC on plastic plates precoated with Matrigel through Western blot analysis of miogenic cells, and FIG. 5b is images showing immunofluorescence staining of the same.

### MODE FOR THE INVENTION

AFSCs are pluripotent stem cells capable of differentiating into multiple lineages, including representatives of the three main embryonic germ layers (ectoderm, endoderm and mesoderm). These cells could be easily mass produced, cryopreserved and shipped to clinics for immediate use as a cell source for therapeutic applications. However, for clinical applications, large amounts of frozen stored cells would be needed and therefore, the development of stem cell banks is necessary. These banks must assure the quality and safety of these cell products, especially when the stored cells are intended for clinical use in cell therapy and regenerative medicine.

During cryopreservation, cell membranes are maximally affected due to intracellular ice formation that takes place during current freezing protocols. Therefore, developing more effective techniques for the cryopreservation of stem cells is an important aspect to consider in order for the banking of cells to become a reality.

Addition of CPAs is a common practice during cryopreservation in order to reduce or eliminate the freezing induced damage to the cells. Currently, most stem cell cryopreservation protocols use the plasma membrane-permeating molecule Me₂SO as a CPA. However, clinical use of frozenthawed stem cells treated with Me₂SO can cause renal failure, cardiac arrhythmias and other complications. Therefore, it is valuable to develop cryopreservation protocols that involve either lower concentrations of Me₂SO or non-toxic CPAs.

Disaccharides, antioxidants and an inhibitor of caspase activity are used in the present invention. The effect of freezing using lower concentrations of Me₂SO in the cryopreservation of AFSCs was analyzed in order to reduce the amount of Me₂SO and eliminate FBS in the infusion product.

Many plants and animals have the ability to survive almost complete dehydration by the accumulation of large amounts of disaccharides, especially sucrose and trehalose. Disaccharides have the ability to form glasses, which have very high viscosity and low mobility, leading to the increased stability of the preserved material. It was suggested that the only requirement for preservation of structure and function in membranes, liposomes, and proteins is the ability of the additive (sugar or polysaccharide) to form a glass. Besides the formation of a glass, a direct interaction between the sugar and polar group in proteins and phospholipids appears to be essential for stabilizing bio-materials of various composition during air drying or freeze drying.

Trehalose, a nontoxic disaccharide of glucose, has been widely used as cryoprotectant as excipients for freeze drying, and stabilizer during dehydration. Its cryoprotective potential has been evaluated in a variety of tissues and cells. The addition of trehalose to Me₂SO-based cryomedia resulted in a high viability rate of cryopreserved pancreas-tissue. It was also shown that when trehalose is used in combination with 10% Me₂SO, it affords better cryoprotection as evidenced by increased colony formation of cryopreserved human hematopoietic stem cells from cord blood and fetal liver as compared to 10% Me₂SO alone.

Another reason of freezing induced damage is the formation of free radicals, which have been implicated as a potential cause of cellular viability loss. Free radicals increased under low moisture and subfreezing conditions results in oxidative damage such as lipid peroxidation, protein oxidation, and DNA damage. The major defense mechanism of cells against free radical-mediated damage includes antioxidants such as ascorbic acid, α-tocopheryl acetate, reduced glutathione, superoxide dismutase, catalase and peroxides.

It was reported that apoptosis plays an important role in the cryoinjury of cells. Apoptosis is induced by activation of both caspase-8 and caspase-9 during cryopreservation. Hence, a synthetic broad-spectrum irreversible caspase inhibitor, ZVAD-fmk is used in combination with other cryoprotectant in order to enhance the post-thaw survival rate of AFSCs.

Because the mode of cryoprotective action of trehalose, catalase and zVAD-fmk are totally different, a combination of these three compounds along with 5% or 2.5% Me₂SO are used in this invention with the aim of reducing the amount of Me₂SO or eliminating FBS in conventional freezing medium.

Preferably, each of trehalose, catalase and ZVAD-fmk is contained at a concentration of 60 mmol/L, 100 mg/mL and 30 μM, respectively, in a conventional freezing medium.

Through the cleavage of the yellow tetrasodium salt MTT to form purple formazan crystal by the metabolic active cells, it was found by the present inventors that frozen cell in the presence of disaccharide (trehalose), antioxidant (catalase) and caspase inhibitor (ZVAD-fmk) in serum free freezing solutions at low concentrations of Me₂SO are more viable after thawing compared with the control solution.

J. P. Rodrigues, F. H. Paraguassu-Braga, L. Carvalho, E. Abdelhay, L. F. Bouzas, L. C. Porto, Evaluation of trehalose and sucrose as cryoprotectants for hematopoietic stem cells of umbilical cord blood, Cryobiology 56 (2008), 144-151 showed that the disaccharides can be used in cryopreservation solutions for hematopoietic stem cells of umbilical cord blood to reduce the concentration of Me₂SO.

L. S. Limaye et al. also demonstrated that antioxidants added in conventional freezing medium improve protection of mouse bone marrow cells and adult human bone marrow.

Further, X. Xu, S. Cowley, C. J. Flaim, W. James, L. Seymour, Z. Cui, The roles of apoptotic pathways in the low recovery rate after cryopreservation of dissociated human embryonic stem cells, Biotechnol. Prog. 26 (2010) 827-837] demonstrate the successful cryopreservation and recovery of cells by using a caspase inhibitor as a cryoprotective agent.

However, none of prior arts concretely discloses the ideal combination for cryopreserving AFSC.

In the present invention, the use of disaccharides, antioxidants and caspase inhibitors for cryopreservation of AFSCs in combination with a lower concentration of Me₂SO is evaluated. The thawed cells are tested for viability with MTT assays and a growth curve is created to measure population doubling time. In addition, cytometry analysis for cell surface antigens, RT-PCR for mRNA expression of stem cell markers, and assays to determine the myogenic differentiation potential of the cells are performed.

According to the present invention, post-thawed AFSCs were found to proliferate quite rapidly. The pattern of growth curve and population doubling time was similar in solutions containing trehalose, catalase and ZVAD-fmk with 5% and 2.5% (v/v) Me₂SO when compared with their controls (5% (v/v) Me₂SO+30% FBS and 2.5% (v/v) Me₂SO+30% FBS).

In immunophenotyping, all viable cells in solutions containing trehalose and catalase with 5% and 2.5% (v/v) Me₂SO expressed markers compatible with a multipotent mesenchymal progenitor lineage. Results of RT-PCR analysis showed that the Oct-4 gene, coding for the transcription factor unique to pluripotent stem cells, was expressed in all solutions we tested. The post-thawed AFSCs were also able to differentiate into the myogenic cells.

According to the present invention, the disaccharide (trehalose), antioxidant(catalase) and caspase inhibitor (ZVAD-fmk) can be used in cryopreservation solutions to reduce the concentration of Me₂SO from current standard 10% (v/v) to 5% (v/v) or 2.5% and to eliminate FBS.

Further, according to the present invention, AFSCs in solutions containing trehalose, catalase and zVAD-fmk with low concentration of Me₂SO can withstand cryopreservation while maintaining their identity and still proliferating rapidly.

Hereinafter, preferred embodiments of the present invention will be described in further detail with reference to Examples. However, it is clearly understood by those of ordinary skill in the art that these Examples are merely to explain the present invention, not to limit the scope of the present invention.

Specimen Collection

AF samples were obtained from amniocentesis performed in the second trimester for routine prenatal diagnosis. The samples were collected after obtaining written informed consent from each patient.

Isolation and primary expansion of AFSCs

AF samples were centrifuged at 1,200 rpm for 10 min. Pellets were resuspended in Chang's medium, which consisted of α-MEM (HyClone, Logan, Utah, USA), Chang's B (Irvine Scientific, Santa Ana, Calif., USA), Chang's C (Irvine Scientific, Santa Ana, Calif., USA), penicillin/streptomycin (HyClone, Logan, Utah, USA), L-glutamine (HyClone, Logan, Utah, USA), embryonic stem (ES)-fetal bovine serum (FBS) (HyClone, Logan, Utah, USA), and the cells were incubated at 37° C. in a humidified atmosphere containing 5% CO₂. After 7 days, non-adherent cells were removed and fresh medium was added. Cells were then cultured for 14 days. The cells were harvested using 0.05% trypsin-EDTA (HyClone, Logan, Utah, USA) for 3 min at 37° C. and replated under the same culture conditions. Thereafter, adherent cells were subcultured when they reached 70% confluence at weekly intervals.

Preparation of Cryoprotectant Solutions

Because Me₂SO is toxic to cells at room temperature, solutions were prepared at 4° C. under sterile conditions. All freezing vials contained 1 ml of final freezing medium after addition of the cells. In all cases, trehalose (Sigma-Aldrich, St. Louis, Mo., USA), catalase (Sigma-Aldrich, St. Louis, Mo., USA) and zVAD-fmk (R&D systems, Minneapolis, Minn., USA) were added to the cryovials at concentrations of 60 mmol/L, 100 μg/mL and 30 μM, respectively. However, different amounts of Me₂SO (2.5%, 5% and 10% (v/v)) were added to the cryovials (Table 1).

TABLE 1
Preparation of different cryoprotectant solutions

| | Me₂SO | FBS | Trehalose | Catalase | zVAD-fmk |
|---|---|---|---|---|---|
| Solution | (% v/v) | (% v/v) | (mmol/L) | (μg/mL) | (μM) |
| 1 | 10 | 30 | 0 | 0 | 0 |
| 2 | 5 | 30 | 0 | 0 | 0 |
| 3 | 5 | 0 | 60 | 100 | 0 |
| 4 | 5 | 0 | 60 | 100 | 30 |
| 5 | 2.5 | 30 | 0 | 0 | 0 |
| 6 | 2.5 | 0 | 60 | 100 | 0 |
| 7 | 2.5 | 0 | 60 | 100 | 30 |

Cryopreservation of AFSCs

Seven different combinations of cryoprotectants were tested with AFSCs (Table 1). A solution of 10% (v/v) Me₂SO+30% FBS was used as the standard cryopreservation solution (100% viability). In all groups, aliquots of 1×10⁶ cells in 100 μL were transferred to 1 mL cryovials containing each cryoprotectant solution. Immediately after the addition of the cells, the cryovials were frozen using a controlled-rate freezer (Cryo, Rockville, Md., USA). All samples were then stored in a liquid nitrogen tank for a minimum of 3 weeks before thawing and further analysis.

Thawing and Secondary Expansion of Cells

Cryopreserved cells were thawed by rapidly immersing the vials in a water bath set at 37° C., and the cells were diluted in the same growth medium as described for isolation and primary expansion. Cells were cultured for 2 passages until the cultures reached the desired cell number. Post-thaw cell viability was assessed by MTT assay. Viable cells were again characterized by flow cytometric analysis of specific surface antigens, construction of growth curves, RT-PCR and an assay for myogenic differentiation potential.

MTT Assay

Aliquots of cell solutions (1×10⁴ cells/mL) were plated with 10 μL MTT solution in each well of a 96-well tissue culture plate and incubated at 37° C. for 4 h. The absorbance of the purple formazan dye formed by the reduction of the MTT reagent was then measured using an ELISA plate reader (Molecular Devices, Sunnyvale, Calif., USA) with a wavelength of 570 nm.

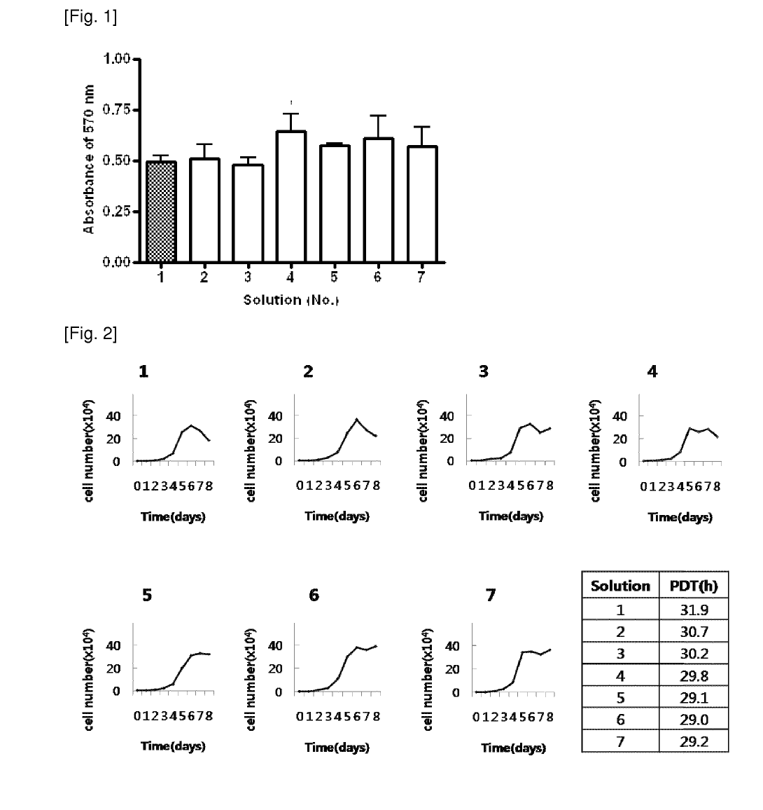

The MTT assay was used to measure cell viability after various freezing conditions. The solutions tested were equivalent or better when compared to the controls (5% (v/v) Me₂SO+30% FBS and 2.5% (v/v) Me₂SO+30% FBS). A statistically significant (p<0.05) increase in post-thaw cell viability when cells were stored in solutions containing trehalose, catalase and zVAD-fmk with 5% Me₂SO was detected (FIG. 1).

Growth Curve (population doubling)

The AFSCs were trypsinized and inoculated into 24-well plates. The cells were incubated at 37° C. with 5% humidified CO₂, and the medium was subsequently replaced three times per week. The cells were counted every 24 h and the mean cell number was recorded as the population cell number for that day. The doubling time of each culture was calculated using these observations.

As expected, AFSCs were found to proliferate rapidly. The growth curve of post-thaw AFSCs in all solutions tested had the following characteristics: (i) in the first 2 days after inoculation the cells adhered to the tissue culture plates; (ii) on day 3, the cells entered the logarithmic growth stage; (iii) peak growth was on day 6; and (iv) the doubling time of the cells in the solutions tested was 29.031.9 h (FIG. 2). No difference in the growth curve was seen when solutions containing trehalose, catalase and zVAD-fmk with 5% and 2.5% (v/v) DMSO were used compared to the controls (5% (v/v) Me₂SO+30% FBS and 2.5% (v/v) Me₂SO+30% FBS) (FIG. 2).

Flow Cytometry Analysis

The specific surface antigens expressed by the cells were characterized by flow cytometry analysis. The post-thaw cells were trypsinized and stained with phycoerythrin (PE)-conjugated human monoclonal antibodies against CD44, CD45, CD73, CD90, CD105, SSEA-4, HLA-ABC and HLA-DR. The cells were analyzed using a flow cytometer (Becton, Dickinson and Co, San Jose, Calif., USA).

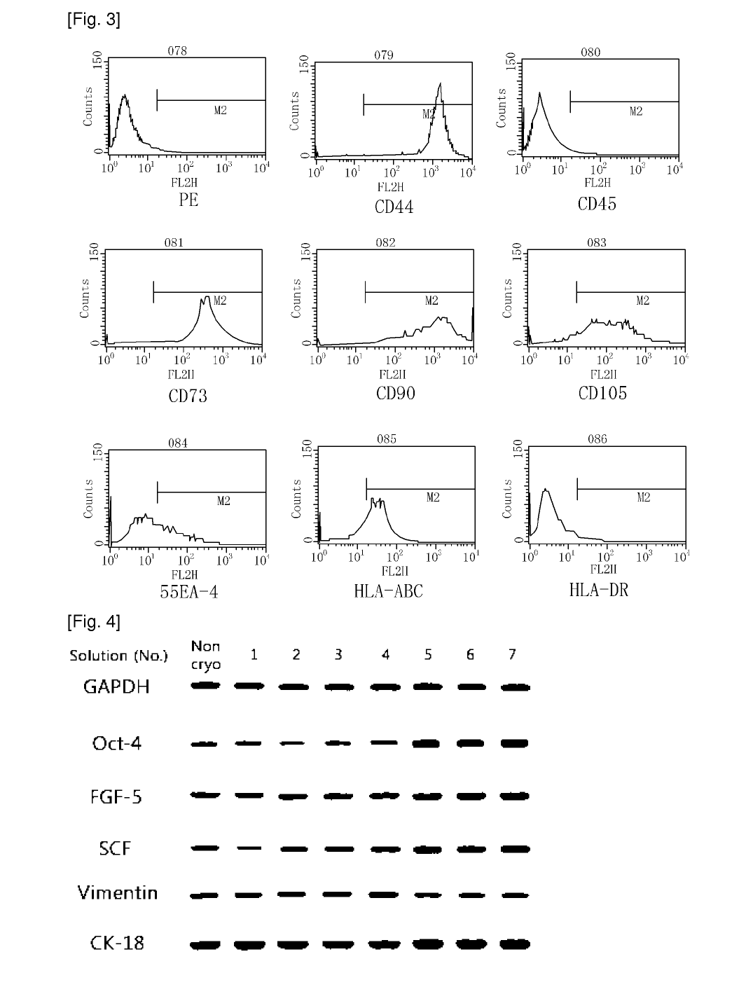

The expression of surface antigens was characterized by flow cytometry using human monoclonal antibodies. The cells in all solutions tested expressed markers compatible with a multipotent mesenchymal progenitor lineage, including CD73, CD90, CD44 and CD105. As expected, these cells were also positive for SSEA-4 and HLA-ABC (MHC class I) but were negative for CD45 and HLADR (MHC class II). There was no difference in the expression of surface antigens in the post-thaw cells that had been stored in solutions containing trehalose and catalase with 5% and 2.5% (v/v) Me₂SO. A representative phenotypic profile of the post-thaw AFSCs is shown in FIG. 3.

Reverse Transcription—Polymerase Chain Reaction (RT-PCR)

Total RNA was extracted from the cultured cells using Trizol reagent (TaKaRa Bio Inc., Shiga, Japan) according to the manufacturer's instructions. RT-PCR was performed using specific DNA primers (Table 2). The amplified DNA fragments were separated using 1% agarose gel electrophoresis, and the bands were stained and photographed under UV light.

TABLE 2
Primers used for RT-PCR

| Gene | Sequence |
|---|---|
| CK-18 | F GAGATCGAGGCTCTCAAGGAR CAAGCTGGC- |
| | CTTCAGATTTC |
| Vimentin | F CCTTCGTGAATACCACGACCTGCR |
| | TAATATATCGCCT-GCCACTGAG |
| FGF-5 | F GCTGTGTCTCAGGGGATTGTAGGAATAR |
| | TATCCAAAGC-GAAACTTGAGTCTGTA |
| SCF | F CCATTGATGCCTTCAAGGACR |
| | CTTCCAGTATAAGGCTCCAA |
| Oct-4 | F ACATGTGTAAGCTGCGGCCR GTTGTG- |
| | CATAGTCGCTGCTTG |
| GAPDH | F GCTTGTCATCAATGGAAATCCCR TCCACACCCAT- |
| | GACGAACATG |

Abbreviations: CK-18: Cytokeratin 18 FGF-5: Fibroblast growth factor 5SCF: Stem cell factorOct-4: Octamer-binding transcription factor 4GAPDH: Glyceraldehyde-3-phosphate dehydrogenase
Analysis by RT-PCR showed that mRNA expression of stem cell markers such as Oct-4, FGF-5, SCF, Vimentin and CK18 was detectable in the post-thaw AFSCs in all solutions tested.

Myogenic Differentiation

AFSCs were seeded on plastic tissue culture plates precoated with Matrigel (BD Biosciences, Bedford, Mass., USA) in DMEM (low-glucose formulation) containing 10% horse-serum (Gibco/BRL, Carlsbad, Calif., USA), 0.5% chick embryo extract (Gibco/BRL, Carlsbad, Calif., USA) and 1% penicillin/streptomycin (HyClone, Logan, Utah, USA). At 12 h after seeding, 5 M 5-aza-2′-deoxycytidine 3 (5-azaC; Sigma-Aldrich, St. Louis, Mo., USA) was added for 24 h. The incubation was then continued in culture medium without 5-azaC, with medium changes every 3 days.

(Western Blot Analysis)

Protein extracts were obtained by treating a plate with 100 μL of lysis buffer (150 mM NaCl, 20 mM TRIS, 1% Triton X-100 and 400 U/mL RNase inhibitor, pH 8) and precipitation with methanol. Forty μg of protein was separated on an SDS-polyacrylamide gel and transferred to nitrocellulose membranes. Sequential incubations with a polyclonal anti-human MyoD (1:500, Santa Cruz Biotechnology, Santa Cruz, Calif., USA) or desmin (1:1000, Cell signaling, Danvers, Mass., USA), secondary peroxidase-conjugated antibody (1:1000, Amersham Bioscience, Uppsala, Sweden) and chemiluminescent kit (ECL, Amersham Bioscience, Uppsala, Sweden) were utilized for specific protein identification.

(Immunofluorescent Staining)

AFSCs were plated in a 24-well plate containing the culture medium described above. The cells were cultured overnight at 37° C. in a 5% humidified CO₂ incubator. The next day, the cells were washed with 10 mM phosphate buffered saline (PBS), pH 7.4, and fixed for 30 min in 2% paraformaldehyde (Sigma-Aldrich, St Louis, Mo., USA) at 4° C. The cells were permeabilized in 0.2% Triton-X (Sigma-Aldrich, St Louis, Mo., USA) for 30 min at room temperature and incubated for a further 20 min in blocking buffer (10% FBS in PBS). The cells were then stained with a primary antibody for either MyoD or desmin (mouse anti-human monoclonal antibodies, Santa Cruz Biotechnology, Santa Cruz, Calif., USA) in PBS for 1 h. Next, 0.5 μg/ml fluorescein isothiocyanate (FITC)-conjugated goat anti-mouse (Santa Cruz Biotechnology, Santa Cruz, Calif., USA) was used as the secondary antibody in PBS for 45 min.

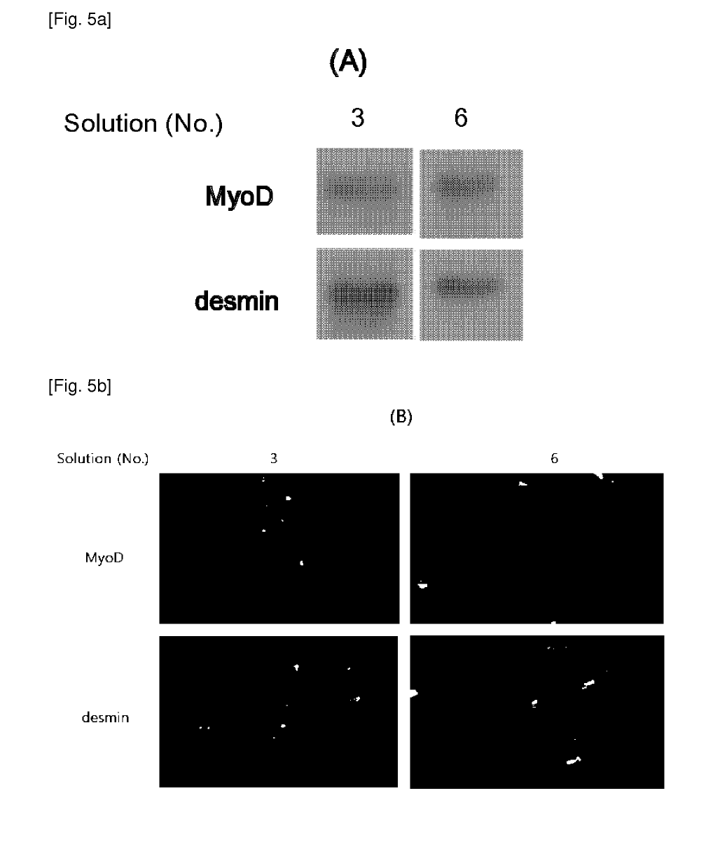

When post-thaw AFSCs were cultured in myogenic medium for 6 days, western blot analysis showed that the AFSCs stored in solutions containing trehalose and catalase with 5% or 2.5% (v/v) Me₂SO expressed Myo D and desmin proteins (FIG. 5a). Likewise, immunofluorescence staining revealed that the AFSCs (in solutions containing trehalose and catalase with 5% or 2.5% (v/v) Me₂SO) expressed Myo D and desmin proteins (FIG. 5b). Taken together, these data confirmed that the post-thaw AFSCs were able to differentiate along the myogenic pathway.

Statistical Analysis

Data are represented as mean±standard deviation. Means were compared using the Student's twotailed t test. A p value of <0.05 was considered to be significant.

1. A freezing medium composition for cryopreserving amniotic fluid-derived stem cells, characterized in that it contains trehalose, catalase and zVAD- fmk(Benzyloxycarbonyl-alyl-alanyl-aspartyl-(O-methyl)-fluoromethyl-ketone) with a lower concentration of Me₂SO.

2. The freezing medium composition according to claim 1, characterized in that the concentration of Me₂SO is 2.5 to 5% (v/v) and a concentration of trehalose is 60 mmol/L and a concentration of catalase is 100 mg/mL and a concentration of zVAD-fmk is 30 μM.

3. A method of cyopreserving amniotic fluid-derived stem cells using the freezing medium composition according to claim 1.

4. A method of cyopreserving amniotic fluid-derived stem cells using the freezing medium composition according to claim 2.
